(19) United States
(12) Patent Application Publication
(10) Pub. No.: US 2026/0010456 A1
(43) Pub. Date: Jan. 8, 2026

(54) Virtual Software Development Advisor

(71) Applicant: GM GLOBAL TECHNOLOGY OPERATIONS LLC, Detroit, MI (US)

(72) Inventors: Paolo GIUSTO, Brentwood, CA (US); Robert J. GENSLAK, White Lake, MI (US); Kohinoor L. BEGUM, Troy, MI (US); Dana WARMSLEY, Newbury Park, CA (US); Sasha STRELNIKOFF, Seattle, WA (US); Aidan BARBIEUX, Montecito, CA (US); Mia LEVY, Venice, CA (US); Jocelyn REGO, Los Angeles, CA (US); Evelyn KIM, Irvine, CA (US)

(21) Appl. No.: 18/761,720

(22) Filed: Jul. 2, 2024

(51) Int. Cl. G06F 11/36; A61B 5/16; G06F 8/33; G06F 8/35

(52) U.S. Cl. CPC G06F 11/3604; A61B 5/165; G06F 8/33; G06F 9/453

(57) ABSTRACT

A computer system for confirming whether user generated code meets defined coding standards, includes a conversion module, a violation module, and an alert module. The conversion module is configured to receive one or more text documents including defined coding standards and generate an executable computer program code based on the defined coding standards. The violation module is configured to receive the computer program code and user generated code and execute the computer program code to determine whether one or more violations of the defined coding standards are present in the user generated code. The alert module is configured to, in response to the one or more violations being present in the user generated code, generate a notification indicating the one or more violations of the defined coding standards in the user generated code. Other example computer system and methods are also disclosed.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to a virtual software development advisor, and more particularly to confirmation of and advisement regarding user generated code with respect to defined coding standards.

Software developers create programs that help computers systems execute tasks. In doing so, the developers create source code which is translated into machine code for execution. The source code may be created by software developers with a wide range of expertise and training, such as by a novice developer with little training, experience, etc. or by an expert software developer with substantial training, experience, etc. In either case, the software developers are often tasked with creating quality source code that complies with defined coding standards or coding guidelines. The coding standards often provide particular rules that software developers follow when writing code to maintain a uniform codebase.

### SUMMARY

A computer system for confirming whether user generated code meets defined coding standards, includes a conversion module, a violation module in communication with the conversion module, and an alert module in communication with the violation module. The conversion module is configured to receive one or more text documents including defined coding standards and generate an executable computer program code based on the defined coding standards. The violation module is configured to receive the computer program code and user generated code, and execute the computer program code to determine whether one or more violations of the defined coding standards are present in the user generated code. The alert module is configured to, in response to the one or more violations being present in the user generated code, generate a notification indicating the one or more violations of the defined coding standards in the user generated code.

In other features, the computer system further includes a recommendation module configured to receive the user generated code and generate one or more recommendations based on the user generated code and one or more previous coding examples.

In other features, the alert module is configured to receive the one or more recommendations from the recommendation module and generate the notification indicating the one or more recommendations.

In other features, the recommendation module includes a cognitive model trained based on the one or more previous coding examples.

In other features, the cognitive model is configured to identify a completion state of the user generated code, and generate the one or more recommendations based on the one or more previous coding examples at a completion state of previously generated code corresponding to the completion state of the user generated code.

In other features, the identified completion state of the user generated code is a numerical representation.

In other features, the computer system further includes at least one sensor configured to monitor a characteristic of an individual while the individual is generating the user generated code, and a cognitive state module configured to receive the characteristic from the sensor and determine whether the individual is experiencing a degraded cognitive state based on the characteristic and a threshold.

In other features, the cognitive state module is in communication with the alert module, the cognitive state module is configured to generate one or more recommendations in response to the individual experiencing the degraded cognitive state, and the alert module is configured to generate the notification indicating the one or more recommendations.

In other features, the one or more recommendations includes a physical action for the individual or a user interface action for a computing device used by the individual to generate the user generated code.

In other features, the at least one sensor includes an on-body sensor attached to the individual to monitor a physiological characteristic of the individual while the individual is generating the user generated code.

In other features, the at least one sensor includes an off-body sensor remote from the individual to monitor a behavioral characteristic of the individual while the individual is generating the user generated code.

In other features, the threshold is an adjustable threshold specific to the individual.

In other features, the alert module is configured to transmit the notification to a computing device used by an individual to generate the user generated code.

In other features, the notification is a visual notification or an audible notification.

A method for confirming whether user generated code meets defined coding standards, includes receiving one or more text documents including defined coding standards, generating an executable computer program code based on the defined coding standards, receiving user generated code, executing the computer program code to determine whether one or more violations of the defined coding standards are present in the user generated code, and in response to the one or more violations being present, generating a notification indicating the one or more violations of the defined coding standards in the user generated code.

In other features, the method further includes transmitting the notification to a computing device used by an individual to generate the user generated code.

In other features, the method further includes generating one or more recommendations based on the user generated code and one or more previous coding examples.

In other features, generating the notification includes generating the notification indicating the one or more recommendations.

In other features, identifying, with a trained cognitive model, a completion state of the user generated code, and generating the one or more recommendations includes generating the one or more recommendations based on the one or more previous coding examples at a completion state of previously generated code corresponding to the completion state of the user generated code.

In other features, the method further includes receiving, from at least one sensor, a characteristic of an individual while the individual is generating the user generated code, determining whether the individual is experiencing a degraded cognitive state based on the characteristic and a threshold, and generating one or more recommendations in response to the individual experiencing the degraded cognitive state.

In other features, generating the notification includes generating the notification indicating the one or more recommendations.

A non-transitory computer-readable medium storing instructions that, when executed by a control module, cause the control module to receive one or more text documents including defined coding standards, generate an executable computer program code based on the defined coding standards, receive user generated code, execute the computer program code to determine whether one or more violations of the defined coding standards are present in the user generated code, and in response to the one or more violations being present, generate a notification indicating the one or more violations of the defined coding standards in the user generated code.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a block diagram of an example computer system for confirming whether user generated code meets defined coding standards, according to the present disclosure;

FIG. 2 is a block diagram of another example computer system for confirming whether user generated code meets defined coding standards and generating various user recommendations, according to the present disclosure;

FIG. 3 is a flowchart of example method for confirming whether user generated code meets defined coding standards, according to the present disclosure; and

FIGS. 4-5 are flowcharts of example methods for confirming whether user generated code meets defined coding standards and generating various user recommendations, according to the present disclosure.

In the drawings, reference numbers may be reused to identify similar and/or identical elements.

### DETAILED DESCRIPTION

Software developers with a wide range of expertise and training create source code for execution by a computing device. For example, developers may be novice developers with little training, experience, etc., expert developers with substantial training, experience, and other levels therebetween. Regardless of the level of expertise and training, software developers are often required to create high quality source code that complies with defined rigorous coding standards. While expert developers may be able to adhere and evangelize to the rigorous coding standards, novice developers often struggle to do so. In some cases, available tools (e.g., commercial off-the-shelf software learning modules) may be useful for generic assistance. However, such tools are insufficient to support novice developers developing software as they do not include specific coding standards for a particular company (e.g., rules, conventions, etc. specific to a particular company), do not reference specific coding documents for a particular company, and do not filter by a software cognitive load of the developer.

The computer systems and methods according to the present disclosure provide both proactive high quality software development and a reactive software refactoring approach that reduces software complexities for software developers, such as novice developers. For example, and as further explained below, the computer systems and methods herein are used to assist novice software developers in creating quality and standards-compliant software in real time via indicators (e.g., visual indicators, etc.) and relevant suggestions during the coding process. Such tools may be ideal for use by organizations or individuals who are looking to decrease software development time, increase code readability and maintainability, and ensure compliance with a set of (organization-specific) coding standards and preferences. As such, the computer systems and methods herein reduce software complexities, and thus produce high quality software that is testable, maintainable, readable, and extensible.

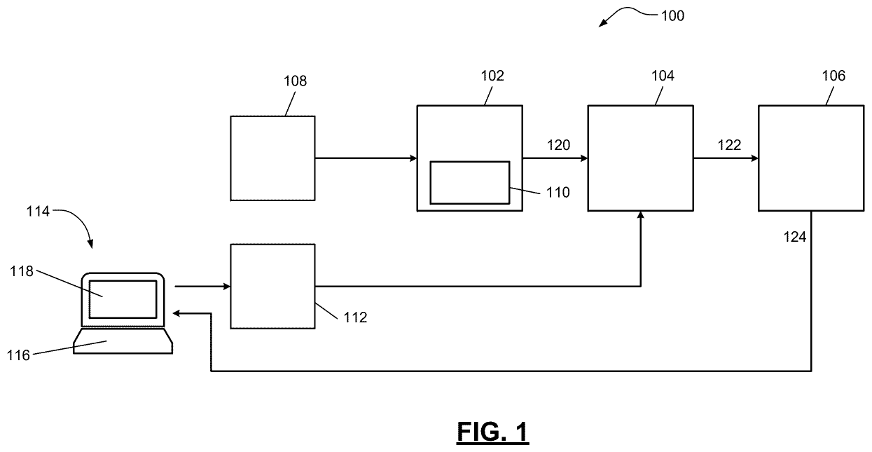

Referring now to FIG. 1, a block diagram of an example computer system 100 is presented for confirming or otherwise verifying whether user generated code meets defined coding standards. As shown in FIG. 1, the computer system 100 generally includes a conversion module 102, a violation module 104, and an alert module 106. As shown, the conversion module 102, the violation module 104, and the alert module 106 are in communication with each other.

Although FIG. 1 illustrates the computer system 100 as including specific dedicated modules, it should be appreciated that one or more other modules may be employed if desired. For example, any combination of the modules (e.g., the conversion module 102, the violation module 104, the alert module 106, etc.) and/or the functionality thereof may be integrated into a single module or multiple different modules.

In various embodiments, the computer system 100 of FIG. 1 enables the development of high-quality software for any type of industries and by all levels of software developers, including novice software developers. As such, the computer system 100 and/or other computer systems and methods herein may be applicable to any suitable industry, including the automotive industry.

With continued reference to FIG. 1, the conversion module 102 and the violation module 104 each generally receives one or more inputs. Specifically, in FIG. 1, the conversion module 102 receives defined coding standards 108. In various embodiments, the defined coding standards 108 may be in the form of one or more text documents and stored in memory. In such examples, the defined coding standards 108 provide particular rules and conventions specific to a particular entity that software developers follow when writing code to maintain a uniform codebase. In the example of FIG. 1, the defined coding standards 108 may be provided to the conversion module 102 via user input, provided to the conversion module 102 in response to a request by the conversion module 102 or another module, pushed to the conversion module 102 (e.g., from another module, memory, etc.), etc.

Additionally, the violation module 104 receives user generated code 112. In such examples, the user generated code 112 may represent a current version of generated code, such as a portion of the code, a finalized version, a draft version, etc. For example, an individual, such as a novice software developer, etc. may utilize one or more user input components (e.g., a keyboard 116, a mouse, etc.) of a computing device 114 to generate the user generated code 112. Then, the computing device 114 may provide the user generated code 112 (e.g., a text file, etc.) to the violation module 104. This may occur automatically at defined time intervals (e.g., periodically), randomly, continuously, etc. In other examples, the computing device 114 may provide the user generated code 112 in response to an input (e.g., user input, a request from the violation module 104, etc.).

In various embodiments, the conversion module 102 may rely on rule-based algorithms and artificial intelligence-based algorithms to transform the text documents of coding standards to a program code that checks for violations of standards. For example, in FIG. 1, the conversion module 102 generates an executable computer program code based on the received coding standards 108. In such examples, the conversion module 102 can automatically convert the coding standards 108 once received. In some examples, the generated executable computer program code may be in a general-purpose programming language, such as Python or another suitable language.

The conversion module 102 may convert the coding standards 108 into the executable computer program code via any suitable manner. For example, and as shown in FIG. 1, the conversion module 102 may include a Large Language Model (LLM) 110 to generate regular expressions (regexes) with the aim of identifying strings (text) in the user generated code 112 that violate the coding standards 108. For example, each regular expression (regex) may include a sequence of characters (e.g., letters, numerical digits, punctuation marks, etc.) that specifies a match pattern in text. In various embodiments, the LLM 110 may be a pretrained model and/or capable of performing in-context learning. In some examples, the generation of the executable computer program code may be accomplished by prompting the LLM 110 with the coding standards 108 along with instructions to generate the relevant regex patterns. In such examples, the coding standards 108 may be required to be in a form that is capturable via the regexes. If the coding standards 108 or another coding standards input into the conversion module 102 is not in a regex capturable form, the conversion module 102 may generate a non-response (e.g., “NA”) if it is not possible to identify a violation using regex patterns.

In various embodiments, examples of violations of the coding standards 108 and required corrections to the examples of violations may be provided to the conversion module 102. In such examples, the conversion module 102 (e.g., the LLM 110) can construct the regexes by identifying the salient difference between the violating and corrected code examples. In some examples, the examples of violations and required corrections may be described in the one or more text documents. In other examples, the examples of violations and required corrections may be labeled examples that are manually constructed and provided directly to the conversion module 102 by a user. Regardless, the constructed regexes can be leveraged directly to identify violating portions of the user generated code 112.

As shown in FIG. 1, the violation module 104 receives an input from the conversion module 102. Specifically, the violation module 104 of FIG. 1 receives executable computer program code (e.g., a text file, etc.) with, for example, the regexes from the conversion module 102. In FIG. 1, the executable computer program code consumed by the violation module 104 is shown by arrow 120. In various embodiments, the violation module 104 may automatically receive the executable computer program code once generated by the conversion module 102. In such examples, new versions of executable computer program code (or a new executable computer program code) may be transmitted to the violation module 104 each time new/updated coding standards are provided to the conversion module 102.

In the example of FIG. 1, the violation module 104 executes the computer program code from the conversion module 102. In doing so, the violation module 104 can determine whether one or more violations of the defined coding standards 108 are present in the user generated code 112. For example, the violation module 104 may monitor (e.g., consistently, routinely, periodically, etc.) a coding process of the individual generating the user generated code 112 (e.g., a novice software developer, etc.), calculating metrics meant to assess the code's readability, maintainability, testability and extensibility and checking for violations of the defined coding standards 108. In various embodiments, the violation module 104 may monitor the user generated code 112 consistently, periodically, and/or in response to an event (e.g., receiving an input signal to proceed, receiving the user generated code, etc.).

In some examples, the violation module 104, via the executed computer program code, is able to scan through the user generated code 112 to identify the one or more violations of the defined coding standards 108 using the regexes. In doing so, the violation module 104 may compute important, well-defined metrics relevant to code quality and development standards. For example, the violation module 104 may compute the number of nested ‘if’ statements, a complexity (e/g/. the number of “&” s and “|”s) of each conditional, a cyclomatic complexity value, Halstead complexity measures (e.g., Halstead difficulty, Halstead complexity, etc.), and source lines of code. In such examples, the violation module 104 may compute metrics of nesting and conditional complexity for each conditional statement, while other metrics are computed for the functions that they are within.

Additionally, in some examples, the violation module 104 may identify duplicate code in the user generated code 112. For instance, the individual generating the user generated code 112 may inadvertently or mistakenly add repetitive sequences that may cause issues with execution and increase processing times, memory requirements, etc. As such, the violation module 104 may identify such instances as, for example, violations of the defined coding standards 108.

In various embodiments, the violation module 104 may utilize the generated computer program code written in a general-purpose programming language (e.g., Python, etc.) with the aid of tree-sitter to complete the determination and identification of violations in the user generated code 112. Accordingly, in this example, tree-sitter is a parser generator and incremental parsing library that provides the ability to map from uncompiled code directly to a Python-parsable abstract syntax tree (AST) representation. In such examples, the use of tree-sitter may be beneficial because it can natively handle partially written code (e.g., the user generated code 112) and is extensible to a large number of popular coding languages. In other examples, another suitable parser generator program and/or parsing library may be employed if desired.

In some examples, the use of the AST representation is useful to remedy other possible complications. In some embodiments, language model techniques of constructing the regexes based on examples of violations and required corrections (with the conversion module 102) may be error prone because key phrases may be used in different contexts within a codebase. For example, the word “continue” includes specific meanings for some particular programming languages but also may be part of phrases used in variable definitions. For instance, “continue” has a specific functional meaning in the C programming language, but is often part of a phrase that could be used in a variable definition (e.g., “bool shouldContinue=true;”). To overcome such issues, the violation module 104 may parse the user generated code 112 (e.g., the complete code or partially written code) into its AST and identify the lowest node in the tree containing the matching statement for the regular expression (regex). The type of this lowest node can be used to provide a restricted search space when leveraging the regex to identify occurrences of violations with an improved accuracy. For example, if the coding standards 108 is intended to restrict the names of variables, the violation module 104 can search for the matches to the regex among only those strings which have the “variable name” node type.

After constructing the AST for the user generated code 112, the violation module 104 executes the computer program code (e.g., the constructed Python functions) to identify violations of each of the coding standards 108 by iterating over each node in the AST and returning the line and character numbers as coordinates to any violating parent-child relationships. Once a violation is identified, the violation module 104 may generate a description of the violation, a line number of the violation, and if applicable, the reference to where the violation is discussed in the coding standards 108. This may be repeated for each identified violation. The violation module 104 may then transmit this information (or least the description of the violation(s)) to the alert module 106. In FIG. 1, the information relating to each violation of the coding standards 108 transmitted to the alert module 106 is shown by arrow 122.

In the example of FIG. 1, the alert module 106 actively captures the coding process of the individual (e.g., the novice software developer) generating the user generated code 112 and sends relevant data to the respective components for analysis. For example, in response to the one or more violations being present in the user generated code 112 and identified by the violation module 104, the alert module 106 may generate a notification indicating each violation of the defined coding standards 108 in the user generated code. In various embodiments, the notification may be generated in real time and/or also include other relevant data, such as a description of the violation, a line number of the violation, the reference to where the violation is discussed in the coding standards 108, etc.

In various embodiments, the alert module 106 may highlight each identified violation of the defined coding standards 108 for the individual. For example, the alert module 106 may transmit the notification to the computing device 114 used by the individual to generate the user generated code 112. In FIG. 1, this transmission of the notification is shown by arrow 124. In such examples, the notification may be a visual notification, an audible notification, and/or another suitable type. For example, the alert module 106 may generate a user interface provided with the notification and/or the computing device 114 may generate a user interface based on the notification highlighting each identified violation (and other relevant data if desired). This user interface may then be rendered on a display 118 of the computing device 114. In some examples, the user generated code 112 may be displayed on the user interface with portions highlighted that correspond to identified violations. In other examples, the alert module 106 may generate an audible message provided with the notification and/or the computing device 114 may generate an audible message based on the notification highlighting each identified violation (and other relevant data if desired). The computing device 114 may then output the audible message via one or more speakers.

In some examples, the alert module 106 may generate the same or another notification indicating each identified violation and/or other previous violations the individual's user generated code. In such examples, the alert module 106 may transmit such information to a human resources department, one or more software developers, one or more supervisors, etc. for review.

In various embodiments, the alert module 106 and/or the functions thereof may be integrated into a software application. For example, in some embodiments, the alert module 106 and/or its functions may be part of an integrated development environment (IDE) accessible and viewable by the individual generating the user generated code 112. In such examples, the IDE may update a visualization interface to display pertinent information, such as violation details and associated guidelines, to the individual. In some examples, the IDE may display identified violations as markers within the interface. These markers can link to a pane/window of the IDE relating to possible issues (e.g., sometimes called a “Problems” pane/window), where more information may be provided, including references to the defined coding standards 108.

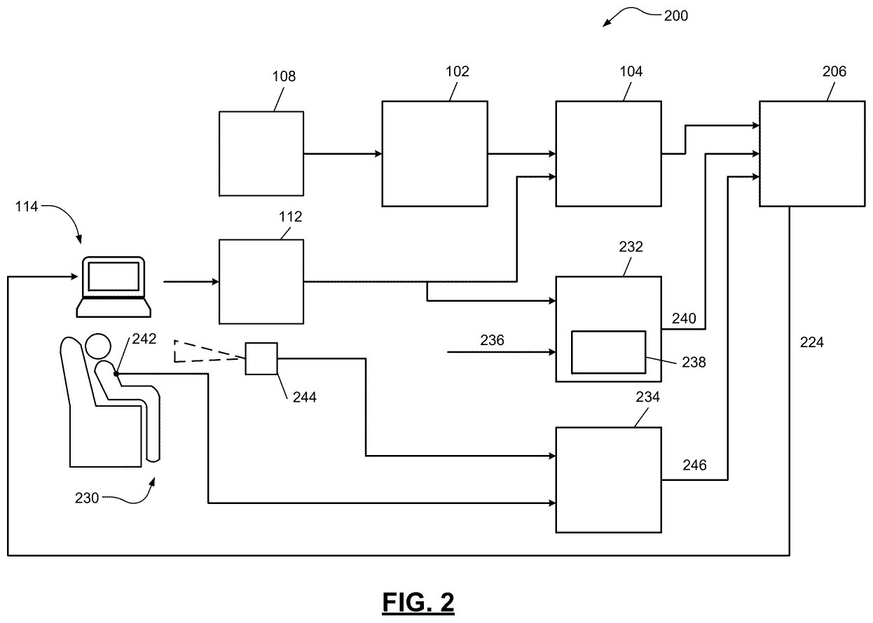

FIG. 2 depicts another example computer system 200 similar to the computer system 100 but including additional components and functionality. For example, and as shown in FIG. 2, the computer system 200 includes the conversion module 102 and the violation module 104 of FIG. 1, each having the same functionality as explained above relative to FIG. 1. For example, the conversion module 102 receives the defined coding standards 108 and generates executable computer program code based on the defined coding standards 108, and the violation module 104 receives the executable computer program code and the user generated code 112 from the computing device 114 (and generated by an individual 230), and indented violations of the defined coding standards 108 in the user generated code 112. As shown, the computer system 200 further includes an alert module 206 similar to the alert module 106 of FIG. 1, a recommendation module 232 and a cognitive state module 234. The recommendation module 232 and the cognitive state module 234 are in communication with the alert module 206.

In the example of FIG. 2, the recommendation module 232 generally provides recommendations based on past coding examples of individuals, such as expert software developers. In some examples, the recommendations may be generated by the recommendation module 232 in response to a user request from the individual 230, a period of inactivity of the individual 230, etc.

For example, and as shown in FIG. 2, the recommendation module 232 receives the user generated code 112 and one or more previous coding examples (represented with arrow 236), and then generates one or more recommendations based on the user generated code 112 and the previous coding examples. In such examples, the recommendation module 232 may then transmit the recommendations to the alert module 206. In FIG. 2, this transmission of the recommendations to the alert module 206 is shown by arrow 240. In doing so, the recommendation module 232 may provide the individual 230 directed guidance (e.g., with a specific issue, code line, etc.) for the user generated code 112 based on past coding examples made by expert software developers the same or similar issue, code line, etc.

In various embodiments, the recommendation module 232 includes a trainable cognitive model 238. In such examples, the cognitive model 238 may be trained based on the previous coding examples and/or other expert coding processes. For example, the trainable cognitive model 238 may learn from experts previous coding processes to make suggestions to the individual 230 (e.g., a novice software developer, etc.) when the individual 230 is faced with similar code scenarios (e.g., similar code that needs refactoring).

As one example, the cognitive model 238 may be trained using an Adaptive Control of Thought-Rational (ACT-R) cognitive architecture with expert refactoring behaviors. Such training may occur offline and/or while the recommendation module 232 is operating. For example, the cognitive architecture integrates cognitive mechanisms for perception and memory to create a mental model of a human, allowing it to perform tasks in the same way as humans. In such examples, the cognitive architecture leverages instance-based learning (IBL), which captures the tendency of humans (e.g., expert software developers) to make decisions by generalizing over similar past experiences characterized by decision-related features, the decision, and outcome utility.

For example, the cognitive model 238 or more generally the recommendation module 232 may identify a completion state of the received user generated code 112. In some examples, the completion state may be a numerical representation. This numerical representation (e.g., a set of values) may represent the current state of the code 112. For instance, the numerical representation may be generated based on a number of lines in the generated code 112, a complexity of the code 112, etc.

The cognitive model 238 may then generate the one or more recommendations based on the previous coding examples at a completion state of previously generated code corresponding to the completion state of the user generated code 112. For example, the recommendation module 232 may receive and store previous coding examples and corresponding numerical representations that represent states of the previously generated code. In some examples, the previous coding examples and corresponding numerical representations may be stored in an expert knowledge base. This provides ties to actions taken by, for example, expert software developers and numerical representations of the previously generated code. Then, given a numerical representation of the current state of the user generated code 112, the cognitive model 238 can identify and recommend actions that an experienced software developer might take by matching that numerical representation to those stored in the expert knowledge base and retrieving associated expert actions. In various embodiments, to obtain the numerical representation necessary, embeddings from code-specific Large Language Models (LLMs) may be extracted, which offer a rich numerical representation of the code that can be used in conjunction with, for example, the generated metrics explained above.

In various embodiments, the previously user generated code may be analyzed by a trained model to identify the previous coding examples and corresponding numerical representations. For example, a LLM (e.g., CodeReviewer, etc.) can be trained based on code including the previously generated code. In doing so, the model can be trained on code edit histories and pull-request reviews that assess quality and suggest changes in the form of comments and new code. In such examples, this model may be pre-trained on a large corpus of text, encoding public collective knowledge on code quality and refactoring suggestions in multiple programming languages.

In such examples, multiple benefits arise from relying on previous coding examples to provide recommendations. For example, the cognitive model 238 can be trained over time as more expert software developers interact with the computer system 200. Additionally, one can control what knowledge is captured by the cognitive model 238 by selectively controlling which users/experts the model learns from. For example, if a company has its own company-specific set of best practices, that company might employ experts in these practices to interact with the computer system 200 for training purposes. This ensures that the learned expert behaviors used to make recommendations to, for example, novice software developers will be aligned with company coding preferences. Further, the cognitive model 238 can be integrated into a company's coding environment and source control service to continuously update the model with expert knowledge, update the model with new techniques of numerical code representation, etc.

With continued reference to FIG. 2, the cognitive state module 234 may generally estimate user workload/stress based on behavioral and physiological patterns from data from one or more sensors and/or user input components (e.g., a mouse, keyboard, etc.). In such examples, sensed data (e.g., brain activity, metabolic activity, heart activity, eye activity, etc.) and/or patterns of mouse and keyboard manipulations may correspond to cognitive states of the individual 230. For example, and as shown FIG. 2, the computer system 200 further includes sensors 242, 244 for monitoring characteristics of the individual 230 while the individual 230 is generating the user generated code 112. The cognitive state module 234 receives the characteristics from at least the sensor 242, 244. While the computer system 200 is shown with two sensors 242, 244, it should be appreciated that the computer system 200 may include more or less sensors of any suitable type. For example, the computer system 200 may only rely on the sensor 244 and keyboard/mouse inputs if desired.

In the example of FIG. 2, the sensor 242 may be an on-body sensor attached to the individual 230 to monitor a physiological characteristic of the individual 230. In such examples, the on-body sensor may include an electroencephalography (EEG) sensor, a functional near-infrared spectroscopy (fNIRS) sensor, a galvanic skin response sensor, a heat rate sensor, a heat rate variability sensor, etc. Additionally, the sensor 244 may be an on off-body sensor remote from the individual 203 to monitor a behavioral characteristic of the individual 230. Specifically, in FIG. 2, the sensor 244 is an eye tracker (e.g., a camera) to monitor eye gaze, head position, pupil size, etc.

Then, the cognitive state module 234 can determine whether the individual 230 is experiencing a degraded cognitive state based on the received characteristics and a threshold. In other words, based at least on the received characteristics, the cognitive state module 234 may determine the individual's cognitive state (e.g., a state of mind, a load level, a stress level, etc.). For example, the cognitive state module 234 may store a baseline value for a characteristic sensed by each sensor 242, 244 (and/or any other behavioral and/or physiological sensor in the computer system 200). This baseline value may be a threshold for that sensor. For instance, the individual 230 can optionally initiate a cognitive test that increases in cognitive load throughout a defined task (e.g., N-Back task, etc.) so that the cognitive state module 234 may obtain baseline measures of the different sensors 242, 244 to determine what high cognitive load looks like specific to the individual 230. In various embodiments, the baseline values (e.g., the thresholds) may be adjusted or updated by performing another cognitive test at a later time.

In various embodiments, the cognitive state module 234 may generate one or more recommendations in response to the individual 230 experiencing the degraded cognitive state. For example, the cognitive state module 234 may recommend a physical action for the individual 230, a user interface action for a computing device 114, etc. For instance, the cognitive state module 234 may suggest that the individual 230 take a break, stand up, stop working, etc. In other examples, the cognitive state module 234 may suggest the highlighting of certain areas on a user interface of the computing device 114 to focus the individual 230, change organization of the user interface, simplify the user interface, etc. Regardless of the type of recommendation, the cognitive state module 234 may transmit the recommendations therefrom to the alert module 206, as shown by arrow 246.

As shown in FIG. 2, the alert module 206 receives the recommendation(s) from the recommendation module 232, the recommendation(s) from the cognitive state module 234, and information related to each identified violation from the violation module 104. Then, in response, the alert module 206 generates and transmits a notification to the computing device 114. In such examples, the notification (e.g., shown by arrow 224) may be similar to the notification of FIG. 1 as explained above. In various embodiments, the notification generated by the alert module 206 may indicate the recommendations provided by the recommendation module 232, indicate the recommendations provided by the cognitive state module 234, and/or each violation of the defined coding standards 108 as identified by the violation module 104. In response, the computing device 114 may alter its user interface and/or notify the individual 230 of the recommendations/violations as explained above.

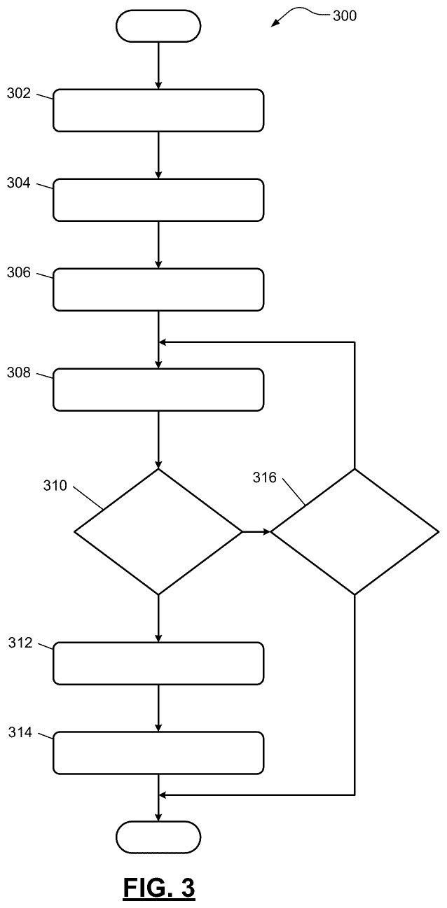

FIGS. 3-5 illustrate example methods 300, 400, 500 employable by the computer system 100 and/or the computer system 200 of FIGS. 1 and 2 for confirming whether user generated code meets defined coding standards. Although the example methods 300, 400, 500 are described in relation to the computer system 100 and/or the computer system 200, any one of the methods 300, 400, 500 may be employable by another suitable computer system.

As shown in FIG. 3, the method 300 begins by receiving one or more text documents with defined coding standards. For example, and as explained above, the defined coding standards (e.g., the defined coding standards 108) may be received by a conversion module, such as the conversion module 102. The method 300 then proceeds to 304, where the conversion module generates executable computer program code based on the defined coding standards. For example, and as explained above, the conversion module 102 may rely on a LLM to generate regexes based on the defined coding standards. The method 300 then proceeds to 306, 308.

At 306, user generated code is received by a violation module, such as the violation module 104. This code may represent a current version of code generated by an individual (e.g., a software developer). Then, at 308, the violation module executes the computer program code generated by the conversion module to determine if violations of the defined coding standards are present in the user generated code.

The method 300 then proceeds to 310. If any violation is present in the user generated code, the method 300 proceeds to 312, where an alert module (e.g., the alert module 106 of FIG. 1 or the alert module 206 of FIG. 2) generates a notification indication each identified violation of the defined coding standards, as explained above. The method 300 then proceeds to 314, where the alert module transmits the notification to, for example, computing used by the individual. The method 300 may then end, as shown in FIG. 3.

If, however, no violations of the defined coding standards are present in the user generated code at 310, the method 300 proceeds to 316. At 316, a determination is made as to whether any update has been made to the user generated code. For example, the violation module may determine if another version of the user generated code has been received. If yes, the method 300 returns to 308, where the violation module executes the computer program code to determine if violations of the defined coding standards are present in the updated user generated code. If no at 316, the method 300 may end, as shown in FIG. 3.

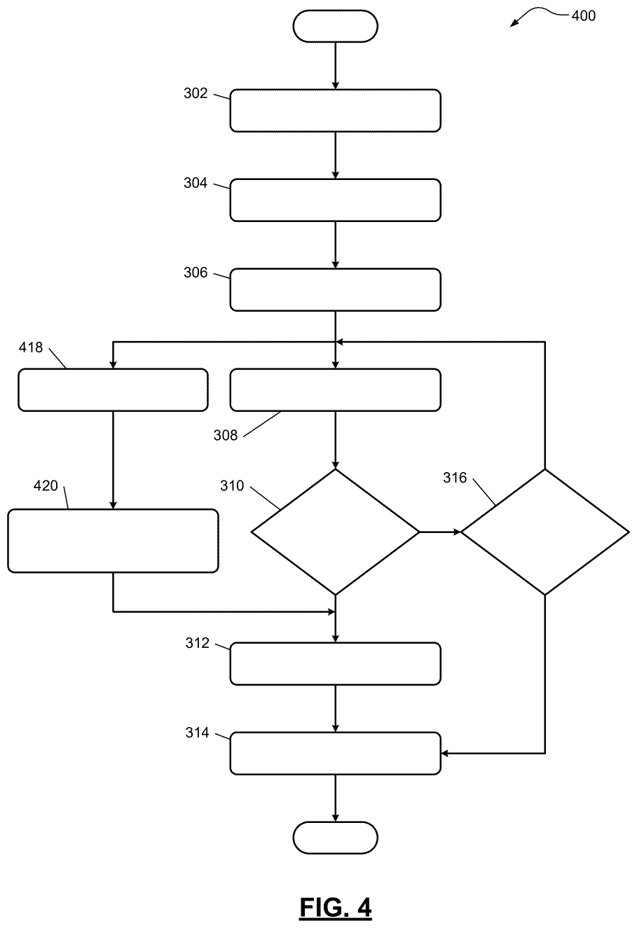

In FIG. 4, the method 400 is similar to the method 300 of FIG. 3 but includes additional steps. For example, and as shown in FIG. 4, the method 400 begins at 302 and then proceeds to 304, 306, 308, 310, 312, 314, 316 as explained above relative to FIG. 3. Additionally, after the user generated code is received by the violation module at 306, the method 400 proceeds to 418, 420.

At 418, a completion state of the received user generated code is identified. For example, and as explained above, a recommendation module (e.g., the recommendation module 232) may include a cognitive model (e.g., the cognitive model 238) that identifies a numerical representation representing the current state of the received user generated code. The method 400 then proceeds to 420.

At 420, the recommendation module generates one or more recommendations based on the identified completion state of the user generated code and previous coding examples. For example, and as explained above, the recommendation module may receive and store previous coding examples and corresponding numerical representations that represent states of previously generated code by, for example, expert software developers. Then, given a numerical representation of the current state of the user generated code, the cognitive model can recommend actions that an experienced software developer took by matching that numerical representation to those stored and retrieving associated expert actions. The recommendation module may then present the expert actions as recommendations. The method 400 then proceeds to 312, 314 where a notification indication each identified violation of the defined coding standards and recommendations may be generated (at 312) and transmitted (at 314), as explained above.

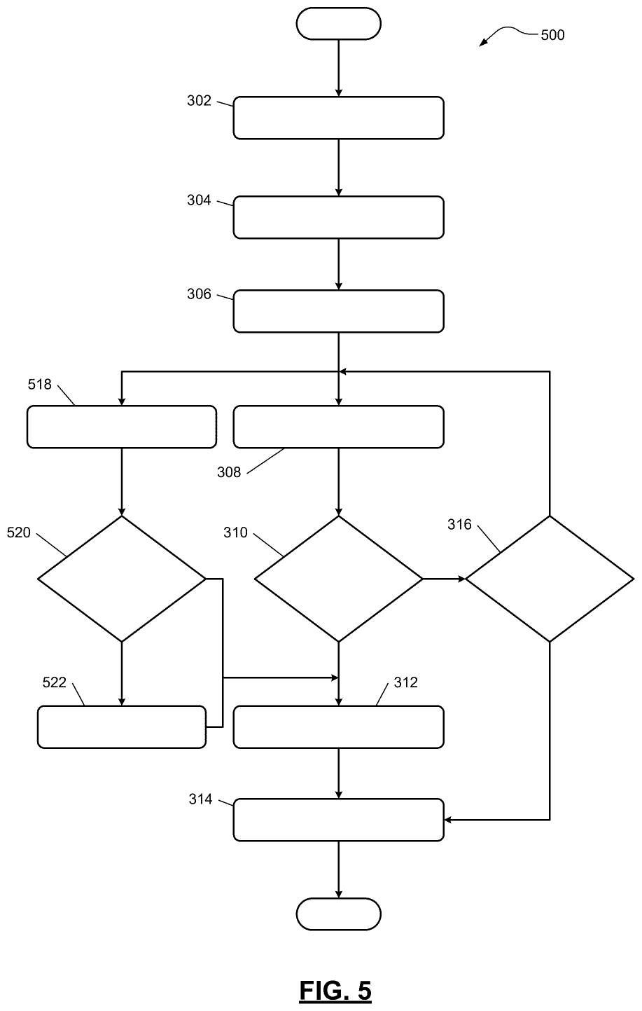

In FIG. 5, the method 500 is similar to the method 300 of FIG. 3 but includes additional steps. For example, and as shown in FIG. 5, the method 500 begins at 302 and then proceeds to 304, 306, 308, 310, 312, 314, 316 as explained above relative to FIG. 3. Additionally, after the user generated code is received by the violation module at 306, the method 500 proceeds to 518, 520, 522.

At 518, one or more characteristics associated with an individual are received by a cognitive state module, such as the cognitive state module 234. For example, and as explained above, the cognitive state module may receive behavioral and physiological characteristics from on-body and/or off-body sensors (e.g., the sensors 242, 244) and/or from user input components (e.g., a mouse, keyboard, etc.) of a computing device used by the individual. The method 500 then proceeds to 520.

At 520, the cognitive state module determines whether the individual is experiencing a degraded cognitive state based on the received characteristics at 518 and one or more thresholds. For example, and as explained above, the cognitive state module may have baseline values (e.g., thresholds) for different sensors, which maybe compared to the received characteristics to determine if the individual is experiencing a degraded cognitive state (e.g., a degraded state of mind, an increased load level, an increased stress level, etc.). If the cognitive state module determines that the individual is not experiencing a degraded cognitive state at 520, the method proceeds to 312. Otherwise, if the cognitive state module determines that the individual is experiencing a degraded cognitive state at 520, the method proceeds to 522.

At 522, the cognitive state module generates one or more recommendations for the individual. For example, the cognitive state module may recommend a physical action for the individual (e.g., stand up, take a break, etc.) and/or a user interface action for the individual's computing device (e.g., highlight certain areas on the user interface, reorganize objects on the user interface, simplify the user interface, etc.). The method 500 then proceeds to 312, 314 where a notification indication each identified violation of the defined coding standards and recommendations may be generated (at 312) and transmitted (at 314), as explained above.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer system for confirming whether user generated code meets defined coding standards, the computer system comprising:
a conversion module configured to receive one or more text documents including defined coding standards and generate an executable computer program code based on the defined coding standards;
a violation module in communication with the conversion module, the violation module configured to receive the computer program code and user generated code, and execute the computer program code to determine whether one or more violations of the defined coding standards are present in the user generated code; and
an alert module in communication with the violation module, the alert module configured to, in response to the one or more violations being present in the user generated code, generate a notification indicating the one or more violations of the defined coding standards in the user generated code.

2. The computer system of claim 1, further comprising a recommendation module configured to receive the user generated code and generate one or more recommendations based on the user generated code and one or more previous coding examples.

3. The computer system of claim 2, wherein the alert module is configured to receive the one or more recommendations from the recommendation module and generate the notification indicating the one or more recommendations.

4. The computer system of claim 2, wherein the recommendation module includes a cognitive model trained based on the one or more previous coding examples.

5. The computer system of claim 4, wherein the cognitive model is configured to:
identify a completion state of the user generated code; and
generate the one or more recommendations based on the one or more previous coding examples at a completion state of previously generated code corresponding to the completion state of the user generated code.

6. The computer system of claim 5, wherein the identified completion state of the user generated code is a numerical representation.

7. The computer system of claim 1, further comprising:
at least one sensor configured to monitor a characteristic of an individual while the individual is generating the user generated code; and
a cognitive state module configured to receive the characteristic from the sensor and determine whether the individual is experiencing a degraded cognitive state based on the characteristic and a threshold.

8. The computer system of claim 7, wherein:
the cognitive state module is in communication with the alert module;
the cognitive state module is configured to generate one or more recommendations in response to the individual experiencing the degraded cognitive state; and
the alert module is configured to generate the notification indicating the one or more recommendations.

9. The computer system of claim 8, wherein the one or more recommendations includes a physical action for the individual or a user interface action for a computing device used by the individual to generate the user generated code.

10. The computer system of claim 7, wherein the at least one sensor includes an on-body sensor attached to the individual to monitor a physiological characteristic of the individual while the individual is generating the user generated code.

11. The computer system of claim 7, wherein the at least one sensor includes an off-body sensor remote from the individual to monitor a behavioral characteristic of the individual while the individual is generating the user generated code.

12. The computer system of claim 7, wherein the threshold is an adjustable threshold specific to the individual.

13. The computer system of claim 1, wherein the alert module is configured to transmit the notification to a computing device used by an individual to generate the user generated code.

14. The computer system of claim 13, wherein the notification is a visual notification or an audible notification.

15. A method for confirming whether user generated code meets defined coding standards, the method comprising:
receiving one or more text documents including defined coding standards;
generating an executable computer program code based on the defined coding standards;
receiving user generated code;
executing the computer program code to determine whether one or more violations of the defined coding standards are present in the user generated code; and
in response to the one or more violations being present, generating a notification indicating the one or more violations of the defined coding standards in the user generated code.

16. The method of claim 15, further comprising transmitting the notification to a computing device used by an individual to generate the user generated code.

17. The method of claim 15, wherein:
the method further includes generating one or more recommendations based on the user generated code and one or more previous coding examples; and
generating the notification includes generating the notification indicating the one or more recommendations.

18. The method of claim 17, wherein:
identifying, with a trained cognitive model, a completion state of the user generated code; and
generating the one or more recommendations includes generating the one or more recommendations based on the one or more previous coding examples at a completion state of previously generated code corresponding to the completion state of the user generated code.

19. The method of claim 15, wherein:
the method further includes receiving, from at least one sensor, a characteristic of an individual while the individual is generating the user generated code, determining whether the individual is experiencing a degraded cognitive state based on the characteristic and a threshold, and generating one or more recommendations in response to the individual experiencing the degraded cognitive state; and
generating the notification includes generating the notification indicating the one or more recommendations.

20. A non-transitory computer-readable medium storing instructions that, when executed by a control module, cause the control module to:
receive one or more text documents including defined coding standards;
generate an executable computer program code based on the defined coding standards;
receive user generated code;
execute the computer program code to determine whether one or more violations of the defined coding standards are present in the user generated code; and
in response to the one or more violations being present, generate a notification indicating the one or more violations of the defined coding standards in the user generated code.
